(19) United States
(12) Patent Application Publication
(10) Pub. No.: US 2015/0164198 A1
(43) Pub. Date: Jun. 18, 2015

(54) Tweezers Needle Wand

(71) Applicant: Keith Andre Mosley, Atlanta, GA (US)

(72) Inventor: Keith Andre Mosley, Atlanta, GA (US)

(21) Appl. No.: 14/634,686

(22) Filed: Feb. 27, 2015

(51) Int. Cl. A45D 26/00; A61B 17/34

(52) U.S. Cl. CPC A45D 26/00; A61B 17/34

(57) ABSTRACT

The Tweezers Needle Wand includes an elongated casing with a tweezers compartment, in which a stationary or removable tweezers resides, on one end and a needle compartment, in which a stationary or removable needle resides, on the opposite end that are covered by protective caps, which are removed to gain access to the tweezers and the needle for the purpose of removing body hair.

### REFERENCES CITED

### U.S. Patent Document

| | | |
|---|---|---|
| 3,971,386 | July 1976 | Yamada . . . 504/403 |
| 4,020,846 | May 1977 | Stokes . . . 128/305 |
| 6,159,222 | December 2000 | Yiu . . . 606/133 |
| 5,072,982 | December 1991 | Boss . . . 294/103.1 |
| 4,994,061 | Febuary 1991 | McPherson . . . 606/133 |
| 6,179,847 | January 2001 | Possum . . . 606/131 |

This invention relates to a hair removal device and, more particular an instrument providing the functionality of tweezers, a needle and protective storage.

### BACKGROUND OF THE INVENTION

Tweezers and a needle have commonly been used to remove ingrown hairs. They are usually stored in separate locations and are not always readily available and cannot be found in a timely manner for use in freeing and removing ingrown hairs. The small diameter of the needle challenges the finger dexterity of most users. The slenderness of the needle requires constant pressure to be applied by the forefinger and thumb in order to hold the needle stationary. This constant pressure results in finger fatigue and begins to impair the steadiness of the hand, which is critical to preventing trauma to the skin surrounding the ingrown hair. Many times picking a needle up from a flat surface can prove to be difficult, not to mention the surface germs the needle is exposed to while not in use. The tweezers consist of two legs joined at one end and possessing a variety of shaped tips at the opposite end; and are usually held between the thumb and forefinger. Tweezers are ideal for removing ingrown hair, unwanted facial and small amounts of body hair. The most popular tweezers tips feature a point, square, round-shaped and slant tip. Slant-tip tweezers are the most common because they are easier and safer to use on ingrown hairs, eyebrows and other facial hairs. Dropping tweezers can dull the point and result in misalignment, rendering them useless. Tweezers should be stored in a protective casing to protect them and extend their useful life.

The object of the present invention is to overcome the disadvantages of the known prior art arrangement, aforementioned inconveniences and risks of damage and exposure to germs by providing a needle and tweezers storage compartment conveniently housed in a protective casing where the tweezers storage compartment and needle are located on opposite ends and are covered by protective caps. The needle's protective cap is removed when it needs to be used to gently tease a trapped hair out of the skin. The tweezers' protective cap is removed in order to gain access to affixed tweezers or retrieve removable tweezers from the storage compartment. The diameter of the invention facilitates the maneuverability. The protective caps prevent the tweezers and needle from being damaged if the instrument is dropped accidently. The present invention is not only ideal for use in the privacy of the home; it is a perfect addition to grooming and cosmetic accessories that are used when traveling.

Although the invention is illustrated and described in relationship to specific embodiments, it is nevertheless not intended to be limited to the details shown, since various modifications and structural changes may be made therein without departing from the spirit of the invention and within the scope and range of equivalents of the claims.

The construction and operation of the invention, however, together with additional objects and advantage thereof will be best understood from the following description of specific embodiments when read in connection with the accompanying drawings.

### SUMMARY OF THE INVENTION

This Tweezers Needle Wand includes a elongated shaped protective case with a storage compartment for tweezers on one end and a needle on the opposite end that are covered by protective caps, which are removed to gain access to the tweezers and to expose the needle.

### BRIEF DESCRIPTION OF THE DRAWINGS

FIG. 1 is a perspective view of the tweezers needle wand according to a first embodiment of the present invention shown with tweezers storage compartment protective cap and needle protective cap engaged.

FIG. 2 is a perspective view of the tweezers needle wand according to a second embodiment of the present invention showing the protective caps removed, exposing the tweezers affixed to one end of the cartridge and a needle affixed to the opposite end, and finger grips along the surface of the cartridge.

FIG. 3 is a perspective view of the tweezers needle wand according to a third embodiment of the present invention showing the protective caps removed, exposing the opening to the removable tweezers storage compartment on one end and a needle affixed to the opposite end of the cartridge, and finger grips along the surface of the cartridge. Tweezers appearing in FIG. 3 is shown for illustrative purposes only and forms no part of the claimed invention therefore no item number has been assigned.

FIG. 4 is a perspective view of the tweezers needle wand according to a fourth embodiment of the present invention showing the protective caps removed, exposing the opening to the removable tweezers storage compartment on one end and a removable needle storage compartment on the opposite end of the cartridge, and finger grips along the surface of the cartridge. Tweezers and needle appearing in FIG. 4 are shown for illustrative purposes only and forms no part of the claimed invention therefore no item number has been assigned.

### DETAILED DESCRIPTION OF THE PREFERRED EMBODIMENTS

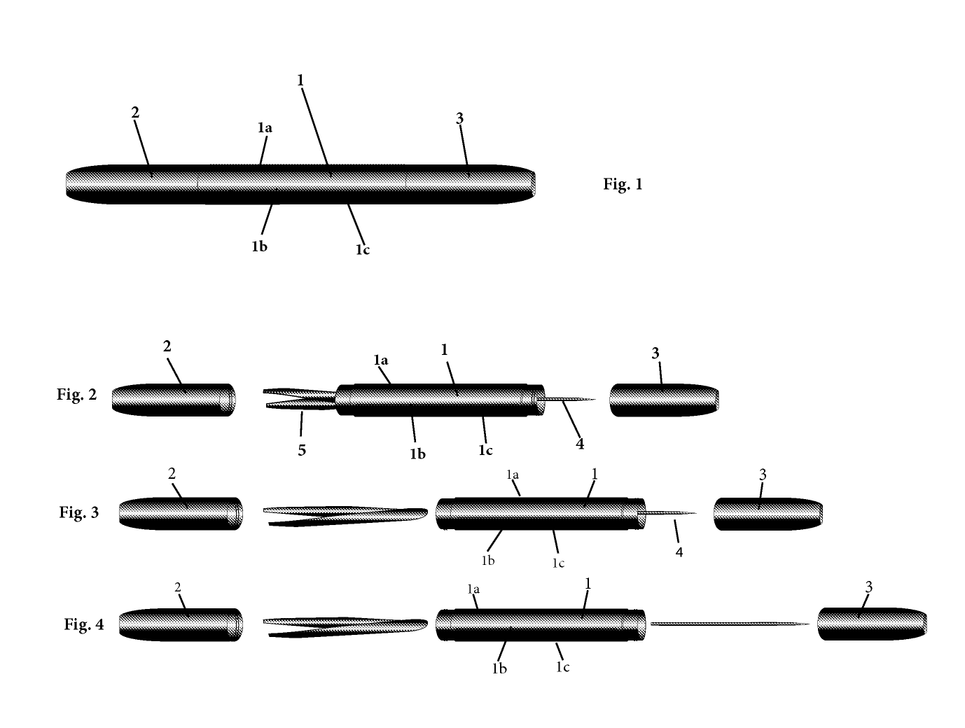

Referring to drawings in detail, wherein like numerals indicate like elements throughout, there is shown in FIGS. 1 through 4 preferred embodiments of an ingrown hair and hair removal device, to be referred to as the Tweezers Needle Wand going forward, in accordance with the present invention. The present invention is shaped to fit comfortably within the hand of the user and to enhance the maneuverability of the device. However, it is within the spirit and scope of the invention to construct the Tweezers Needle Wand of any conventional shape conducive to being comfortably hand held, such as an eyebrow pencil or other such hand held cosmetic instrument.

Referring to FIG. 1, the Tweezers Needle Wand consisting of an elongated casing 1 covered with finger grips 1a-1c, 1d is located on backside, with tweezers protective cap 2 and needle protective cap 3 engaged. A tweezers storage compartment protective cap 2 engages one end of housing 1, via twist-on/off or snap-on/off technology, for the purpose of closing the tweezers storage compartment. A needle protective cap 3 engages the opposite end of casing 1, via twist-on/off or snap-on/off technology, for the purpose of covering the needle 4 when not in use.

Referring to FIG. 2, the Tweezers Needle Wand consisting of an elongated casing 1 covered with finger grips 1a-1c, 1d is located on backside, with tweezers protective cap 2 detached, exposing the tweezers 5 affixed to one end of casing 1, and needle protective cap 3 detached, exposing the needle 4 affixed to the opposite end of casing 1. A tweezers storage compartment protective cap 2 engages one end of housing 1, via twist-on/off or snap-on/off technology, for the purpose of closing the tweezers storage compartment. A needle protective cap 3 engages the opposite end of casing 1, via twist-on/off or snap-on/off technology, for the purpose of covering needle 4 when not in use.

Referring to FIG. 3, the Tweezers Needle Wand consisting of an elongated casing 1 covered with finger grips 1a-1c, 1d is located on backside, with tweezers protective cap 2 detached, exposing the removable tweezers storage compartment at one end of casing 1, and needle protective cap 3 detached, exposing needle 4 affixed to the opposite end of casing 1. A tweezers storage compartment protective cap 2 engages one end of housing 1, via twist-on/off or snap-on/off technology, for the purpose of closing the removable tweezers storage compartment. A needle protective cap 3 engages the opposite end of casing 1, via twist-on/off or snap-on/off technology, for the purpose of covering needle 4 when not in use.

Referring to FIG. 4, the Tweezers Needle Wand consisting of an elongated casing 1 covered with finger grips 1a-1c, 1d is located on backside, with tweezers protective cap 2 detached, exposing the removable tweezers storage compartment at one end of casing 1, and needle protective cap 3 detached, exposing the removable needle 4 located at the opposite end of casing 1. A tweezers storage compartment protective cap 2 engages one end of housing 1, via twist-on/off or snap-on/off technology, for the purpose of closing the removable tweezers storage compartment. A needle protective cap 3 engages the opposite end of casing 1, via twist-on/off or snap-on/off technology, for the purpose of covering removable needle when not in use.

What is claimed:

1. A tweezers needle wand that allows for the use and storage of tweezers and needle for the purpose of removing ingrown or unwanted hair, comprising:
(a) a tweezers wand comprised of an elongated casing with either tweezers affixed to one end or a storage compartment in which a pair of removable tweezers can be stored.
(b) a needle either affixed to the opposite end of the casing or a storage compartment in which a removable needle can be stored.
(c) removable protective caps that cover the tweezers and needle when not in use.
